(19) United States
(12) Patent Application Publication
(10) Pub. No.: US 2007/0104692 A1
(43) Pub. Date: May 10, 2007

(54) Breast tissue regeneration

(76) Inventors: Rodolfo C. Quijano, Laguna Hills, CA (US); Hosheng Tu, Newport Beach, CA (US); Kenneth J. Williams, Brawley, CA (US); Robert L. Carter, Joplin, MO (US); Alexander Kiselyov, Del Mar, CA (US)

Correspondence Address: HOSHENG TU, 15 RIEZ, NEWPORT BEACH, CA, 92657-0116, US

(21) Appl. No.: 11/268,392

(22) Filed: Nov. 7, 2005

(51) Int. Cl. A61K 35/12; C12N 5/08

(52) U.S. Cl. 424/93.7; 435/366

(57) ABSTRACT

A stem-cell-seeded porous scaffold implant and methods for treating a breast tissue defect in a patient.

### FIELD OF THE INVENTION

The present invention is related to stem cells for treatment of breast tissue defect, more particularly, the present invention relates to stem-cell-seeded porous scaffold or matrix as an implant to repair or augment a breast tissue defect in a patient.

### BACKGROUND OF THE INVENTION

It was reported that adipose-derived stem cells might be engulfed in injured heart muscle following a heart attack-like injury. Adipose, also known as fat tissue, contains a specialized class of stem cells, which are comprised of multiple cell types that might promote healing and repair. It appears that adipose-derived stem cells home in on specific sites of injury through biological signaling that occurs naturally during heart attacks.

In addition to pluripotent stem cells of embryonic origin, several groups described mammalian multipotent stem cell populations that are obtained from adult somatic cell sources. Non-embryonic multipotent stem cells include, for example, neural stem cells, mesenchymal stem cells, bone marrow stem cells and stem cells obtained from liposuction. It is important to note that the adult multipotent stem cells described in the prior art have limited potential, in that they have not been demonstrated to give rise to any and all cell types of the body. In general, a stem cell shows ability of a clonal stem cell population to self-renew, ability of a clonal stem cell population to generate a new, terminally differentiated cell type in vitro and ability of a clonal stem cell population to replace an absent terminally differentiated cell population when transplanted into an animal depleted of its own natural cells.

Mesenchymal stem cells are adult multipotent cells derived from multiple sources, including bone marrow stroma, blood, dermis, and periosteum. These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells (U.S. Pat. No. 5,736,396). It was reported that mesenchymal cells, upon injection into either mouse or rat brains, are capable of migrating through the brain, engrafting, surviving, and differentiating into astrocytes, ependymal cells, or neurons, suggesting the capacity of mesenchymal stem cells to give rise to cells of a non-mesenchymal lineage (U.S. Pat. No. 5,197,985, U.S. Pat. No. 5,226,914, U.S. Pat. No. 5,486,359, and U.S. Pat. No. 5,736,396).

U.S. Pat. Nos. 6,429,013 and 6,841,150, entire contents of which are incorporated herein by reference, discloses pluripotent stem cells generated from adipose tissue-derived stromal cells and uses thereof. Specifically, the patents disclose that an isolated adipose tissue derived stromal cell is induced to express at least one characteristic of a neuronal cell, an astroglial cell, a hematopoietic progenitor cell, and a hepatic cell. Further, the patents discloses a method for dedifferentiating isolated adipose tissue-derived stromal cells, comprising: plating the isolated adipose tissue-derived stromal cells at a density of approximately 1,000 to 500,000 cells/cm² and incubating the cells in medium comprising i) serum; ii) at least one compound selected from the group consisting of: growth factors, hormones, cytokines and serum factors; and iii) optionally, an embryonic extract.

Important parts of the breasts include mammary glands, the axillary tail, the lobules, Cooper's ligaments, the areola and the nipple. As breasts are mostly composed of adipose tissue, their size can change over time if the woman gains or loses weight. Adipose tissue is an anatomical term for loose connective tissue composed of adipocytes. Its main role is to store energy in the form of fat, although it also cushions and insulates the body. It has an important endocrine function in producing hormones such as leptin, resistin and TNFα. It also functions as a reserve of nutrients. Adipose tissue has an “intracellular matrix,” rather than an extracellular one. Adipose tissue is divided into lobes by small blood vessels. The cells of this layer are adipocytes.

Recent advances in biotechnology have allowed for the harvesting of adult stem cells from adipose tissue, allowing stimulation of tissue regrowth using a patient's own cells. The use of a patient's own cells reduces the chance of tissue rejection.

Five stages of breast development include: a) the first childhood stage: the breasts are flat and show no signs of development; b) the second breast bud stage: milk ducts and fat tissue form a small mound; c) the third breast growth stage: breast become rounder and fuller; d) the fourth stage with nipple and areola forming separate small mound: not all girls go through this stage; and e) the firth stage: breast growth enters finial stage showing an adult breast full and round shaped. For those women with breast defect, it is desirable to transplant stem cells or stem-cell-seeded porous scaffold as an implant to repair or augment the breast tissue defect.

Whereas embryonic stem cells are the building blocks for all of the cell types in the body, adult stem cells are a more specialized type of progenitor cell. Adult stem cells are found in specific tissues and have the ability to regenerate themselves, as well as differentiate into all of the cell types found in that tissue. The specific differentiation pathway that these cells enter depends upon various influences from mechanical influences and/or endogenous bioactive factors, such as growth factors, cytokines, and/or local microenvironmental conditions established by host tissues. Using cells from the developed individual, rather than an embryo, as a source of autologous or allogeneic stem cells would overcome the problem of tissue incompatibility associated with the use of transplanted embryonic stem cells, as well as solve the ethical dilemma associated with embryonic stem cell research.

Adipose tissue offers a potential source of multipotential stromal stem cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively non-invasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells which are capable of self-renewal.

### SUMMARY OF THE INVENTION

One object of the invention is to provide a method and compositions for directing adipose-derived stromal cells cultivated in vitro to differentiate into breast tissue progenitor cells for implantation into a recipient for the therapeutic treatment of pathologic conditions in breast tissue.

Some aspects of the invention relate to a method of providing stem cells for treatment of breast tissue defect. In one preferred embodiment, the method comprises providing stem-cell-seeded porous scaffold or construct as an implant to repair or augment a breast tissue defect in a patient. The adipose-derived stem cells home in on specific sites of breast defect or injury through biological signaling that occurs naturally for a breast defect or pathologic conditions.

Some aspects of the invention relate to a method of providing stem cells for cosmetically modifying breast tissue, wherein the method comprises providing stem-cell-seeded scaffold or construct as an implant to cause breast tissue defect due to implantation and providing breast tissue regeneration through stem cells of stem-cell-seeded scaffold or construct for repairing or augmenting the breast tissue defect in a patient.

Some aspects of the invention relate to a method of treating a breast defect in a patient, the method comprising differentiating an isolated human adipose tissue derived stromal cell into a breast tissue progenitor cell and administering the breast tissue progenitor cell to a breast defect area in the patient. In one embodiment, the progenitor cell further comprises a biocompatible shaped matrix or scaffold, wherein the biocompatible matrix may be non-biodegradable or biodegradable. In a further embodiment, the biodegradable matrix may be made of a material selected from a group consisting of polymers or copolymers of lactide, glycolide, caprolactone, polydioxanone, trimethylene carbonate, polymers or copolymers of polyorthoesters and polyethylene oxide, and polymers or copolymers of aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, and hydrogel. In a further embodiment, the biocompatible matrix comprises a material selected from a group consisting of alginate, agarose, fibrin, collagen, methylcellulose, and combinations thereof.

In one embodiment, the breast defect is traumatically created by any of the following conditions or processes: inserting the biocompatible matrix into the patient, lumpectomy, mastectomy, breast reconstruction, breast injury, or other breast surgical procedures.

In an alternative embodiment, the progenitor cell further comprises a biocompatible cell carrier, wherein the cell carrier may be in a form selected from a group consisting of slurry, gel, colloid, solution, or suspension that is flowable. In one embodiment, the cell carrier or gel is malleable. Further, the cell carrier is selected from a group consisting of alginate, agarose, fibrin, collagen, chitosan, gelatin, elastin, and combinations thereof. In one embodiment, the biocompatible cell carrier is biodegradable.

Some aspects of the invention relate to a method of treating a breast defect in a patient, the method comprising differentiating an isolated human adipose tissue derived stromal cell into a breast tissue progenitor cell and administering the breast tissue progenitor cell to a breast defect area in the patient, wherein following administration of the progenitor cell to a breast defect area in the patient, the progenitor cell further differentiates in situ in the patient.

Some aspects of the invention provide a composition for treating a breast defect of a patient, comprising stem cells derived from adipose tissue and a temperature-sensitive cell carrier, wherein the stem cells may comprise breast tissue progenitor cells. In one embodiment, the temperature-sensitive cell carrier is methylcellulose, poly(N-isopropyl acrylamide), or the like. In one embodiment, the temperature-sensitive cell carrier is characterized by a first solution phase at a lower temperature and a second gel phase at a higher temperature. In another embodiment, the temperature-sensitive cell carrier is characterized by an expanded conformation at a lower temperature and a collapsed conformation at a higher temperature. In a further embodiment, the composition is a compressible foam, a shaped scaffold, a porous matrix or flowable/malleable material.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawing.

FIG. 1 shows a schematic diagram of a method for treating a breast defect.

### DETAILED DESCRIPTION OF THE EXEMPLARY EMBODIMENTS

The preferred embodiments of the present invention described below relate particularly to methods and a composition for the differentiation and culture of adipose tissue-derived stromal cells into breast tissue progenitor cells. The cells produced by the methods of the invention are useful in providing a source of fully differentiated and functional cells for tissue regeneration for the treatment of human breast defect, repair and augmentation. Thus, in one aspect, the invention provides a method for differentiating adipose tissue-derived stromal cells into breast tissue progenitor cells comprising culturing stromal cells in a composition which comprises a medium capable of supporting the growth and differentiation of stromal cells into functional progenitor cells. This invention further provides methods for the introduction and position of these stromal cells in breast defect areas for repair or augmentation. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

By “progenitor” it is meant an oligopotent or multipotent stem cell which is able to divide without limit and, under specific conditions, can produce daughter cells which terminally differentiate such as into breast cells. These cells can be used for transplantation into a heterologous, autologous, or non-autologous host. By heterologous is meant a host other than the animal from which the progenitor cells were originally derived. By autologous is meant the identical host from which the cells were originally derived. Cell suspensions in culture medium are supplemented with certain specific growth factor which allows for the proliferation of target progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation or purification of the cells and re-suspension in fresh medium containing specific growth factors. The medium for cells suspension is also considered one type of cell carriers.

By “adipose” is meant any fat tissue. The adipose tissue may be brown or white adipose tissue, derived from subcutaneous, omental/visceral, mammary, gonadal, or other adipose tissue site. A convenient source of adipose tissue is from liposuction surgery, however, the source of adipose tissue or the method of isolation of adipose tissue is not critical to the invention. When stromal cells are desired for autologous transplantation into a subject, the adipose tissue will be isolated from that subject and administered to the specific breast defect site for tissue regeneration.

Any medium capable of supporting stromal cells in tissue culture may be used, for example, Dulbecco's Modified Eagle's Medium that supports the growth of fibroblasts. Growth factors are generally added to the medium for supporting stromal cells in tissue culture. Typically, 0 to 20% Fetal Bovine Serum (FBS) is added to the above medium in order to support the growth of stromal cells. The cells could be incubated at a temperature around 37° C. with the carbon dioxide content maintained between 1% to 10% and the oxygen content between 1% and 20%.

Non-limiting examples of media useful in the methods of the invention can contain fetal serum of bovine or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%.

The growth factors of the invention may include, but not limited to, transforming growth factor-β (TGF-β1, TGF-β2, TGF-β3 and the like), insulin-like growth factor, platelet derived growth factor, epidermal growth factor, acidic fibroblast growth factor, basic fibroblast growth factor, hepatocytic growth factor, and the like. The concentration of growth factors is about 1 to about 100 ng/ml. In one embodiment, the matrix for incorporating the stromal cells is a component of the collagenous extracellular matrix such as collagen I (particularly in the form of a gel). Other nutrient, such as vitamin A, vitamin A analogue (such as retinoic acid), vitamin B series, vitamin C, and vitamin C analogue or other vitamins may be added to the medium. The concentration of retinoic acid or other nutrient is about 0.1 to about 10 μg/ml.

The present invention also provides a method for formulating adipose derived stromal cells, either after in vitro culture or in absence of in vitro culture, with a biocompatible pharmaceutical carrier for injecting into the breast of a subject. In one embodiment, the biocompatible carrier may be in the form of slurry, gel, a malleable gel, colloid, solution, or suspension. A process for manufacturing an implantable cells-seeded gel material may comprise the steps of: providing a biocompatible carrier and stem cells source; combining the cells and the carrier in a uniformly suspended form; and applying a pressurizing force to the combined fluid for either injecting into the breast of the subject or for collapsing into a malleable gel before administering into the breast.

The adipose tissue derived stromal cells useful in the methods of invention may be isolated by a variety of methods known to those skilled in the art. For example, such methods are described in U.S. Pat. No. 6,153,432 incorporated herein in its entirety. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject so as to provide for an autologous transplant. Alternatively, the administered tissue may be allogenic.

In one embodiment of the invention, an adipose tissue derived stromal cell induced to express at least one phenotypic characteristic of a neuronal, astroglial, hepatic, hematopoietic, or breast tissue progenitor cell is provided. Phenotypic markers of the desired cells are well known to those of ordinary skill in the art, and copiously published in the literature. Additional phenotypic markers continue to be disclosed or can be identified without undue experimentation. Any of these markers can be used to confirm that the adipose cell has been induced to a differentiated state. Lineage specific phenotypic characteristics can include cell surface proteins, cytoskeletal proteins, cell morphology, and secretory products. Some aspects of the invention provide adipose tissue-derived stromal cells that exhibit the improved properties of increased extracellular matrix proteins and/or a lower amount of lipid than a mature isolated adipocyte.

Malson et al. in U.S. Pat. No. 4,772,419, entire contents of which are incorporated herein by reference, describes a crosslinked hyaluronic acid (or salt thereof) gel material that may be formed into a shaped article by pressure-drying or freeze-drying. The crosslinked hyaluronic material may be stored dry, and implanted or placed upon a body in dry form, or alternatively after being rehydrated in a saline solution. The crosslinking present in the material causes the material to be rehydrated as a sponge or foam, wherein the structure or shape is maintained, rather than forming a flowable hydrogel or putty. Some aspects of the invention provide a crosslinked gel material as a shaped article loaded with adipose-derived stem cells or progenitor breast tissue cells.

In another embodiment, the biocompatible cell carrier (for example, for cells to home in) or matrix may be a shaped construct, structure, or 3-dimensional scaffold. Examples of biocompatible carrier material includes alginate, agarose, fibrin, collagen, chitosan, gelatin, elastin, and combinations thereof In one embodiment, the biocompatible cell carrier is biodegradable or bioresorbable. Examples of biodegradable matrix material may include, but not limited to, polymers or copolymers of lactide, glycolide, caprolactone, polydioxanone, and trimethylene carbonate. Examples of biodegradable matrix material may also include polyorthoesters and polyethylene oxide.

Further examples of biodegradable polymers for construction of-the matrix may include aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, hydrogel, lactide/tetramethylglycolide copolymers, lactide/trimethylene carbonate copolymers, lactide/ε-capro-lactone copolymers, lactide/σ-valerolactone copolymers, 1-lactide/dl-lactide copolymers, methyl methacrylate-N-vinyl pyrrolidone copolymers, modified proteins, nylon-2 PHBA/-γ-hydroxyvalerate copolymers (PHBA/HVA), PLA/polyethylene oxide copolymers, PLA-polyethylene oxide (PELA), poly (amino acids), poly (trimethylene carbonates), poly hydroxyalkanoate polymers (PHA), poly(alklyene oxalates), poly(butylene diglycolate), poly(hydroxy butyrate) (PHB), poly(n-vinyl pyrrolidone), poly(ortho esters), polyalkyl-2-cyanoacrylates, polyanhydrides, polycyanoacrylates, polydepsipeptides, polydihydropyrans, poly-dl-lactide (PDLLA), polyesteramides, polyesters of oxalic acid, polyglycolide (PGA), polyiminocarbonates, polylactides (PLA), poly-1-lactide (PLLA), polyorthoesters, poly-p-dioxanone (PDO), polypeptides, polyphosphazenes, polysaccharides, polyurethanes (PU), polyvinyl alcohol (PVA), poly-β-hydroxypropionate (PHPA), poly-β-hydroxybutyrate (PBA), poly-σ-valerolact-one poly-β-alkanoic acids, poly-β-malic acid (PMLA), poly-ε-caprolactone (PCL), pseudo-Poly(Amino Acids), starch trimethylene carbonate (TMC), tyrosine based polymers. In another embodiment, the cell carrier or matrix functions as a reservoir for cell differentiation and controlled release to adjacent tissue sites.

Current protocols for differentiating isolated human preadipocytes into adipocytes can be performed by a variety of methods, for example, the preadipocyte cell component in human adipose tissue (the so-called “stromal vascular fraction” or SVF) can be isolated using collagenase treatment. The isolated human preadipocytes can then be driven to differentiate into adipocytes by a variety of chemical treatments. For example, Hauner's laboratory (Journal Clin Invest., (1989) 34:1663-1670) has shown that human preadipocytes can be induced to differentiate in serum-free medium containing 0.2 nM triiodothyronine, 0.5 μM insulin and 0.1 μM glucocorticoid. Similarly, it is disclosed in U.S. Pat. No. 4,153,432, entire contents of which are incorporated herein by reference, for the differentiation of human preadipocytes that incubating isolated human preadipocytes, plated at least about 25,000 cells/cm², in a medium containing, glucose, a cyclic AMP inducer such as isobutylmethylxanthine or forskolin, a glucocorticoid or glucocorticoid analogue, insulin or an insulin analogue and a PPARγ agonist or a RXR agonist.

### EXAMPLE NO. 1

### Methods of Transplantation

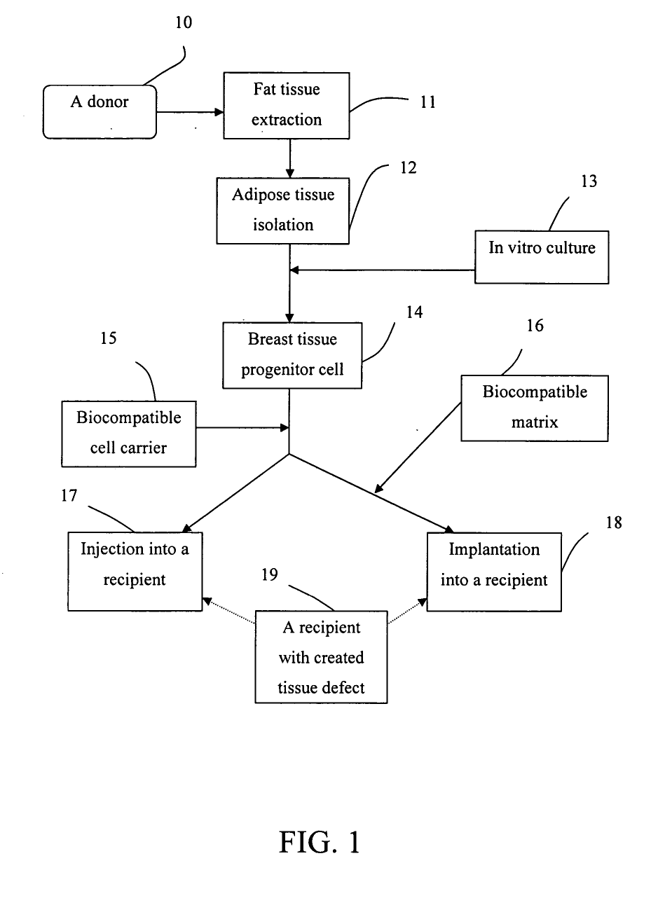

FIG. 1 shows a method of treating a breast defect in a patient, the method comprising: a) differentiating an isolated human adipose tissue derived stromal cell into a breast tissue progenitor cell; and b) administering the breast tissue progenitor cell to a breast defect area in the patient. In one embodiment, the fat tissue from the donor is further differentiated into adipocytes in an in vitro procedure, followed by isolation to obtain a concentrated substance of breat tissue progenitor cells prior to the step of administering. In one embodiment, the breast tissue defect is created as an adjunct step for promoting stem cells differentiation and tissue regeneration at about the defect site.

As shown in FIG. 1, the fat tissue extraction step 11 may be carried out, for example by liposuction from a donor 10. The adipose tissue isolation step 12 may include breakup of the fat mass and removal of the unwanted non-cellular material. In vitro culture step 13 may be optional; however, nutrients, growth factors and other substance may be added to enhance cell differentiation into breast tissue progenitor cells. In one embodiment, the breast tissue progenitor cells 14 can be formulated with biocompatible cell carrier 15 for injection into a recipient 17. In another embodiment, the breast tissue progenitor cells 14 can be further deposited onto a biocompatible matrix 16 for implantation into a recipient 18. It is one object of the present invention to provide a recipient 19 with created tissue defect enabling the stem cells tissue regeneration via the injection route 17 or the implantation route 18.

In another embodiment of the invention, support cells are used to promote the differentiation of the adipose-derived stromal cells prior to or following implantation into the defect breast site of a recipient. The support cells can be human or non-human animal derived cells. Adipose-derived cells are isolated and cultured within a population of cells; most preferably, the population is a defined population. The population of cells is heterogeneous and includes support cells for supplying factors to the progenitor cells of the invention. Support cells include other cell types that will promote the differentiation, growth and maintenance of the desired cells. By way of illustration, adipose-derived stromal cells are first isolated by any of the means described above, and grown in culture in the presence of other support cells. In another embodiment, the support cells are derived from primary cultures of these cell types taken from cultured human organ tissue. In yet another embodiment, the support cells are derived from immortalized cell lines. In some embodiments, the support cells are obtained autologously.

### EXAMPLE NO. 2

### Cell Carriers and Matrix

The formula consisting of breast tissue progenitor cells and cell carriers can be injected to the defect site of the breast using a syringe or other fluid delivery apparatus. In one embodiment, the formula is intended to enhance revascularization in situ. In another embodiment, the formula is intended to promote growth or multiplication of fat cells in the breast. For illustration purposes, the biocompatible matrix for cells to home in or adhere for intended differentiation purposes may comprise a foam or sponge that is compressible for inserting into the breast with a small opening. The biocompatible foam or sponge construct is characterized with plural pores, wherein at least a portion of the pores is interconnected and open to the outside of the construct. The foam or sponge can be cut, sized, and shaped as an implant. In one embodiment, the formula consisting of breast tissue progenitor cells and cell carriers may be loaded on the biocompatible matrix/foam before matrix/foam delivery into a recipient. Alternatively, the formula consisting of breast tissue progenitor cells and cell carriers may be injected to about the matrix/form site after the matrix/foam is implanted in place.

The gel or foam of the present invention may comprise methylcellulose, a temperature-sensitive polymer. Methylcellulose (MC) is a water-soluble polymer derived from cellulose, the most abundant polymer in nature. As a viscosity-enhancing polymer, it thickens solutions without precipitation over a wide pH range. A novel method using a temperature-sensitive polymer (Methylcellulose) to thermally gel aqueous alginate blended with distinct salts (CaCl₂, Na₂HPO₄, or NaCl), as a pH-sensitive hydrogel was developed for protein drug delivery (Biomacromolecules 2004;5:1917-1925). In the preparation of cells loaded hydrogels herein, it is suggested that stem cells is well-mixed to the dissolved aqueous methylcellulose or methylcellulose/alginate blended with salts at 4° C. and then gel by elevating the temperature to 37° C. In one embodiment, the blend (stem cells or adipose-derived breast tissue progenitor cells plus aqueous methylcellulose) is injected into the breast of a recipient and become a gel in situ because of the body temperature at 37° C., a characteristic temperature for methylcellulose.

All methylcellulose compositions exhibit the classical physical behavior of cellulose ethers, changing from a solution at lower temperature to a gel at elevated temperatures. When exposing methylcellulose to an increasing temperature, the methylcellulose shows an initial period of relatively constant viscosity. Then the solution undergoes an abrupt increase in viscosity at a characteristic temperature corresponding to initiation of the first gelation phenomenon. The temperature at which gelation is initiated can be altered by varying a number of factors, including concentration of methylcellulose polymer, formulation of the aqueous solvent, additives, and heating rate. Methylcellulose was reported biocompatible with little toxicity due to degraded byproducts (Biomaterials 2001;22:1113-1123). It was reported that injectable methylcellulose appears to be a suitable scaffold for bridging traumatically injured tissue when a cavity forms within the first few days following a traumatic insult to the cortex.

Poly(N-isopropyl acrylamide) demonstrated a fully expanded chain conformation below 32° C. and a collapsed compact conformation at high temperatures (J Biomed Mater Res 1993;27:1243-1251). In one aspect of the invention, adipose-derived breast tissue progenitor cells or stem cells are mixed with poly(N-isopropyl acrylamide) to form an injectable gel. material. After loading the gel material into the breast of a recipient at adjacent the porous scaffold, the gel material collapses and squeezes into the pores of the scaffold, where the stem cells start differentiation and proliferation to repair or treat breast tissue defect.

Although the present invention has been described with reference to specific details of certain embodiments thereof, it is not intended that such details should be regarded as limitations upon the scope of the invention. Many modifications and variations are possible in light of the above disclosure.

What is claimed is:

1. A method of treating a breast defect in a patient, the method comprising:
a) differentiating an isolated human adipose tissue derived stromal cell into a breast tissue progenitor cell; and
b) administering said breast tissue progenitor cell to a breast defect area in the patient.

2. The method of claim 1, wherein said progenitor cell further comprises a biocompatible matrix or scaffold.

3. The method of claim 2, wherein said biocompatible matrix is biodegradable.

4. The method of claim 3, wherein said biodegradable matrix is made of a material selected from a group consisting of polymers or copolymers of lactide, glycolide, caprolactone, polydioxanone, and trimethylene carbonate.

5. The method of claim 3, wherein said biodegradable matrix is made of a material selected from a group consisting of polymers or copolymers of polyorthoesters and polyethylene oxide.

6. The method of claim 3, wherein said biodegradable matrix is made of a material selected from a group consisting of polymers or copolymers of aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, and hydrogel.

7. The method of claim 2, wherein the breast defect is traumatically created by a process of inserting said biocompatible matrix into the patient.

8. The method of claim 2, wherein said biocompatible matrix comprises a material selected from a group consisting of alginate, agarose, fibrin, collagen, methylcellulose, and combinations thereof.

9. The method of claim 1, wherein said progenitor cell further comprises a biocompatible cell carrier.

10. The method of claim 9, wherein said cell carrier is in a form selected from a group consisting of slurry, gel, colloid, solution, or suspension.

11. The method of claim, 10, wherein said gel is malleable gel.

12. The method of claim 9, wherein said cell carrier is selected from a group consisting of alginate, agarose, fibrin, collagen, chitosan, gelatin, elastin, and combinations thereof.

13. The method of claim 9, wherein said biocompatible cell carrier is biodegradable.

14. The method of claim 1, wherein following administration of said progenitor cell to a breast defect area in the patient, the progenitor cell further differentiates in situ in said patient.

15. A composition for treating a breast defect of a patient, comprising stem cells derived from adipose tissue, and a temperature-sensitive cell carrier.

16. The composition of claim 15, wherein the stem cells comprise breast tissue progenitor cells.

17. The composition of claim 15, wherein the temperature-sensitive cell carrier is methylcellulose.

18. The composition of claim 15, wherein the temperature-sensitive cell carrier is poly(N-isopropyl acrylamide).

19. The composition of claim 15, wherein the temperature-sensitive cell carrier is characterized by a first solution phase at a lower temperature and a second gel phase at a higher temperature.

20. The composition of claim 15, wherein the temperature-sensitive cell carrier is characterized by an expanded conformation at a lower temperature and a collapsed conformation at a higher temperature.
